(19) United States
(12) Patent Application Publication
(10) Pub. No.: US 2008/0096242 A1
(43) Pub. Date: Apr. 24, 2008

(54) Enhanced Substrate Conversion Efficiency Of Fermentation Processes

(75) Inventors: Johan Pieter Marinus Sanders, Groningen (NL); Ruud Alexander Weusthuis, Heelsum (NL); Andreas Mooibroek, Renkum (NL)

Correspondence Address: MORGAN LEWIS & BOCKIUS LLP, 1111 PENNSYLVANIA AVENUE NW, WASHINGTON, DC, 20004, US

(73) Assignee: AGROTECHNOLOGY AND FOOD INNOVATIONS B.V., NL- 6708 PD Wageningen (NL)

(21) Appl. No.: 11/661,548

(22) PCT Filed: Aug. 31, 2005
(86) PCT No.: PCT/NL05/00628

§ 371 Date: Jan. 4, 2008

(30) Foreign Application Priority Data
Sep. 1, 2004 (EP) 04077442.4

(51) Int. Cl. C12P 1/00; C12M 3/00

(52) U.S. Cl. 435/41; 435/289.1

(57) ABSTRACT

The present invention relates to the field of fermentation technology. In particular the invention relates to fermentation processes for the production of a first and a second fermentation product by a single production organism wherein the first product is in a more reduced state than the substrate and the second fermentation product is in a more oxidised state than the substrate yet in a less oxidised state than the final oxidation product CO₂, such that the concurrent synthesis of the first and second product in the organism allows recycling of reducing power and can be performed under (partially) anaerobic conditions. The invention also relates to such processes in which the first and second fermentation products are capable of forming a(n) (insoluble) complex or salt.

### FIELD OF THE INVENTION

The present invention relates to fermentation process for the production of at least a first and a second fermentation product. In these fermentation processes the substrate conversion efficiency is improved by concurrent production of the first and second fermentation products, each of which has a particular oxidation state with respect to each other and with respect to the substrate such that reduction equivalents may be recycled within the production organism, thereby reducing the need for complete oxidation of substrate to CO₂. Further improvements in the fermentation processes of the invention comprises the concurrent production of a weak alkaline compound and a weak acidic compound, and/or the first and a second fermentation products are capable of forming a(n) (insoluble) complex or salt for efficient removal of products. The possible improvements can also be translated into improvements in volumetric productivity.

### BACKGROUND OF THE INVENTION

In the fermentation industry carbohydrates are generally used as raw material, i.e. substrate, which usually is the carbon and energy source, for the fermentative production of compounds having either the same or a more reduced state than the substrate, as is the case with e.g. some amino acids, or having a more oxidised state than the substrate, as is the case with e.g. most organic acids. In fermentation processes in which a compound is produced that has a more reduced state than the carbon and energy source, the production organism requires significant amounts of reducing power. This reducing power is generated by complete oxidation of the substrate to CO₂. Thus, a significant part of the substrate is lost to the production of CO₂, reducing the overall efficiency of the process. Moreover, in the complete oxidation of the substrate to CO₂ usually a surplus reducing power is generated. This surplus reducing power can be used in the conversion of substrate into biomass, and/or used to generate metabolic energy (in the form of ATP or proton motive force). The high availability of metabolic energy again results in the formation of biomass. This conversion of substrate into biomass further reduces the overall efficiency of the process. For respiration the organism requires large amounts of oxygen and produces large amounts of heat. Oxygen transfer capacity and cooling capacity are usually rate limiting in industrial fermentation processes and increasing either of these capacities is either capital intensive or impossible due to physical constraints of the fermenter system. Therefore, there is a need in the art for fermentation processes with improved substrate to product conversion efficiency, higher volumetric production efficiency, reduced heat generation and/or reduced oxygen demand. It is an object of the present invention to provide such improved processes.

### DESCRIPTION OF THE INVENTION

The present invention concerns fermentation processes with enhanced substrate conversion efficiency, higher volumetric production rates, reduced heat generation and/or reduced oxygen demand. In a first aspect of the invention these improved fermentation characteristics are achieved by avoiding and/or reducing that reducing power, required by the organisms for growth and product formation, is generated by complete oxidation of the substrate to CO₂. In many fermentation processes, the final product has a more reduced state, than the substrate that is used for the production of the final product. As result, the organism requires reducing power in the form of NADH, NADPH or FADH. In aerobic organisms reducing power is generated by complete oxidation of the substrate to CO₂, which is not a product of much economic value. The present invention aims to avoid or reduce complete oxidation of substrate to CO₂ but rather to generate sufficient reducing power by incomplete oxidation of substrate to a second product that has an oxidation state that is higher than that of the substrate and at the same time lower than the oxidation state of CO₂. This may be achieved by forcing the production organism into, or by enabling an incomplete oxidation metabolism that may be partially anaerobic. A large number of bacteria, yeasts and filamentous fingi are capable of such incomplete oxidation as is evidenced by their capability to accumulate organic acids. By incomplete oxidation to e.g. organic acids the organism generates NADH or other reducing equivalents, which in turn can be used for the reduction of the substrate (e.g. carbon source) to a first final product having a more reduced state than the substrate, such as e.g. amino acids.

Thus, in a first aspect the invention relates to a method for the production of at least a first fermentation product and at least a second fermentation product by conversion of a substrate by a production organism into the first fermentation product and the second fermentation product, wherein the organism comprises a first metabolic pathway for conversion of the substrate into the first fermentation product and a second metabolic pathway for conversion of the substrate into the second fermentation product, wherein the first fermentation product is in a more reduced state than the substrate and the second fermentation product is in a more oxidised state than the substrate, wherein the first and second pathways are complementary in that their concurrent action in the organism allows recycling of reducing power in the organism, wherein the second fermentation product is in a less oxidised state than the final oxidation product CO₂, wherein the combined yields of the first and second fermentation products on substrate, each on a C-molar basis, is at least 40%.

The method of the invention is a method for producing a fermentation product, whereby a fermentation product is understood to mean any useful compound or group of useful compounds that may be produced by an organism, preferably by a microorganism. The fermentation product may be a final or intermediary metabolite of the organism's metabolism, which metabolism may either be the organism's natural metabolism or a non-natural metabolism that was modified in the organism, e.g. by human intervention. In the context of the invention, fermentation products thus include e.g. carbohydrates, sugars, including mono-, oligo- and polymeric carbohydrates, lipids, fatty acids, amino acids, oligo- and polypeptides, proteins, organic acids, vitamins, intermediary metabolites, secondary metabolites, antibiotics, alkanols, alkohols, polyols, amines, diamines, polyamines. However, in the context of the present invention, explicitly excluded from the terms “first fermentation product” and “second fermentation product” are biomass, CO₂ and energy, usually in the form of heat. In the context of the present invention CO₂ is not considered useful products and a further object of the invention is to reduce to the amounts of biomass and heat produced in the fermentation processes of the invention.

In the method of the invention the substrate preferably is a carbon and energy source for the production organism, preferably the sole carbon and energy source for the organism. In most industrial fermentations, carbohydrates are used as source of carbon and energy for the production organism. Suitable carbohydrate carbon and energy sources are well known in the fermentation art and include e.g. glucose, fructose, lactose, galactose, xylose, arabinose, saccharose, maltose, trehalose, maltodextrine, cellulose, hemicellulose, cellobiose, soluble starch, molasses, corn starch, lignin, lignocellulose, and the like. In the present invention, the oxidation state of the substrate is a reference point with respect to which the oxidation states of the first and second fermentations product are respectively defined. For practical purposes we will however mostly discuss carbohydrate carbon and energy sources in the examples herein. However, by no means is the present invention limited to the use of only carbohydrates as carbon and energy sources. Suitable non-carbohydrate carbon and energy sources include e.g. glycerol, mannitol, citric acid, alkanols (e.g. methanol or ethanol), polyols, lipids, fatty acids, organic acids, amines, amino acids, peptides, alkanes, alkanoates, hydroxy-alkanoates, lignin and its derivatives, and the like.

In the method of the invention, the oxidation states of the first and second fermentation products are defined in relation to the oxidation state of the substrate. The first fermentation product is in the same or a more reduced state than the substrate. Thus, the first fermentation product has the same or a higher redox potential than the redox potential of the substrate. Conversely, the second fermentation product is in a more oxidised state than the substrate whereby a second provision to be fulfilled by the second fermentation product is that it is in a less oxidised state than the final oxidation products CO₂ and H₂O. Thus, the second fermentation product has a lower redox potential than the redox potential of the substrate and the second fermentation product has a higher redox potential than the redox potential of the final oxidation products CO₂ and H₂O. Each of the substrate, first and second fermentation products may consist of a group of different compounds that differ in oxidation state (see also below). In such instances, the redox potential of such a group of compounds is herein defined as the mean redox potential per C-mol.

The choice of the oxidation states of the first and second fermentation products respectively, with respect to the oxidation state of the substrate and the combination in one production organism of the metabolic pathways for conversion of the substrate into the first and second fermentation products, respectively, creates a situation in the organism wherein the concurrent action of the first and second pathways allows recycling of reducing power in the organism. The first and second pathways are thus complementary in that the first pathway requires reducing power, usually in the form of NADH, NADPH or FADH, which reducing power is generated by the second pathway rather than by complete oxidation to the final oxidation products CO₂ and H₂O.

An advantage of the method of the invention is that less of the substrate is converted into CO₂ and more is converted into the first and second fermentation products. Therefore, in a preferred method of the invention, the combined product yields of the first and second fermentation products on substrate, each on a C-molar basis, is at least 30, 35, 40, 50, 60, 70, 80 or 90%. The product yield (or yield coefficient of product over substrate; Y_{PS}) on a C-molar basis is herein understood to mean the amount carbon atoms in product produced per the amount of carbon atoms in substrate utilised. In the method of the invention, the product yields on substrate on a C-molar basis of the first and second products may be substantially equal to each other but in most instances this will not be the case. Therefore at least 51, 52, 55, 60, 65, 70, 75, 80 or 90% of the combined product yields on a C-molar basis of the first and second fermentation products, may consist of either the product yield of the first or of the second fermentation product.

Preferably in the method of the invention, the production organism consumes less than 30, 20, 18, 15, 12, 10, 8 or 5% of the amount of oxygen on a C-molar basis related to the substrate consumed during the conversion of the substrate into the first and second fermentation product. The conversion coefficient of oxygen consumed over substrate utilised on a C-molar basis (C_{OS}) is herein understood to mean mol O₂ used per C-mol substrate consumed. In theory the method of the invention could be carried out under strict anaerobic conditions (i.e. C_{OS}=0.0), and such processes are not excluded from the invention. However, in a preferred method some oxygen is consumed. Such processes may be referred to as partially-, semi-anaerobic processes or micro-aerobic processes that are carried out under low oxygen conditions.

In another preferred embodiment of the method of invention the yield coefficient of biomass produced over substrate utilised is less than 30, 25, 20, 15, 10, 5 or 2%. The yield coefficient of biomass produced over substrate utilised (Y_{XS}) is herein understood to mean the amount of biomass produced in grams dry weight over the amount of substrate utilised in grams.

In one embodiment of method of the invention, the first fermentation product comprises more than one compound. In such instances the average reduction state per C-mol of the compounds in the first fermentation product is higher than the average reduction state per C-mol of the substrate. Preferably, each of the compounds in the first product is in a more reduced state than the substrate. Similarly, in another embodiment, the second fermentation product comprises more than one compound. In such instances the average reduction state per C-mol of the compounds in the second fermentation product is lower than the average reduction state per C-mol of the substrate each of the compounds in the second product is in the same or in a more oxidised state than the substrate.

Suitable examples of first fermentation products to be produced in the method of the invention in the case that the substrate is carbohydrate, and which products thus have the same or a more reduced state than carbohydrate include e.g. amino acids such as lysine, glutamic acid, leucin, threonin, tryptophan; antibiotics, including e.g. ampicilline, bacitracin, cephalosporins, erythromycin, monensin, penicillins, streptomycin, tetracyclines, tylosin, macrolides, and quinolones; lipids and fatty acids including e.g. poly unsaturated fatty acids (PUFAs); alkanol such as ethanol, propanol and butanol; polyols such as 1,3-propane-diol, butandiol, glycerol and xylitol; ketons such as aceton; amines, diamines, ethylene; isoprenoids such as carotenoids, carotene, astaxanthin, lycopene, lutein; acrylic acid, sterols such as cholesterol and ergosterol; vitamins including e.g. the vitamins A, B₂ B₁₂, C, D, E and K; and peptides, including e.g. oligopeptides, polypeptides, proteins including e.g. proteins having industrial or medicinal (pharmaceutical) applications. Examples of proteins or polypeptides with industrial applications include enzymes such as e.g. lipases (e.g. used in the detergent industry), proteases (used inter alia in the detergent industry, in brewing and the like), carbohydrases and cell wall degrading enzymes (such as, amylases, glucosidases, cellulases, pectinases, beta-1,3/4- and beta-1,6-glucanases, rhamnoga-lacturonases, mannanases, xylanases, pullulanases, galactanases, esterases and the like, used in fruit processing, wine making and the like or in feed), phytases, phospholipases, glycosidases (such as amylases, beta.-glucosidases, arabinofuranosidases, rhamnosidases, apiosidases and the like), dairy enzymes and products (e.g. chymosin, casein), polypeptides (e.g. poly-lysine and the like, cyanophycin and its derivatives). Mammalian, and preferably human, polypeptides with therapeutic, cosmetic or diagnostic applications include, but are not limited to, collagen and gelatin, insulin, serum albumin (HSA) and lactoferrin.

Suitable examples of second fermentation products to be produced in the method of the invention in the case that the substrate is carbohydrate, and which products thus have a more oxidised state than carbohydrate include e.g. most organic acids that may be produced by fermentation such as glucaric acid, gluconic acid, glutaric acid, adipic acid, succinic acid, tartaric acid, oxalic acid, acetic acid, lactic acid, formic acid, malic acid, maleic acid, malonic acid, citric acid, fumaric acid, itaconic acid, levulinic acid, xylonic acid, aconitic acid, ascorbic acid, kojic acid, and comeric acid. Included are derivatives and salt of the aforementioned acids whereby a derivative of an acid may be any derivative having one or more substitutions at any position except for the carbonyl group.

The skilled person will appreciate that when a substrate with a lower or higher oxidised state of the carbohydrate is used, products will be moved from the of second fermentation products to the group of first fermentation products or vice versa, respectively, depending on whether a particular product has a higher or lower redox than the redox of the new substrate.

In another aspect the invention relates to a method for the production of at least a first fermentation product and at least a second fermentation product by conversion of a substrate by a first production organism into the first fermentation product and by conversion of a substrate by a second production organism into the second fermentation product, wherein the first and second production organisms are co-fermented and wherein the first fermentation product is a weak alkaline compound or a salt thereof or derivative thereof and the second fermentation product is a weak organic acid or a salt thereof or derivative thereof.

In yet another aspect the invention relates to a method for the production of at least a first fermentation product and at least a second fermentation product by conversion of a substrate by a first production organism into the first fermentation product and by conversion of a substrate by a second production organism into the second fermentation product, wherein the first and second production organisms are co-fermented and wherein a first and a second fermentation product are capable of forming an insoluble complex or insoluble salt under the fermentation conditions applied. Preferably, the first and the second fermentation products are capable of forming a non-ideal solution or a complex, preferably an insoluble complex or a salt, preferably an insoluble salt under the fermentation conditions applied. Such conditions include e.g. a pH range between pH 4.0, 5.0 or 6.0 at one end of the range and pH 7.0, 8.0 or 9.0 on the other end of the range and at a concentration 3 M (3 moles/liter), 2 M, 1 M, 0.5 M, 250 mM, 100 mM, 50 mM for each of the weak alkaline or weak acidic compound and in all different combinations. Preferred combinations capable of forming a non-ideal solution or a(n) (insoluble) complex or salt are combinations of the weak acids and weak alkaline compounds. The production of non-ideal solutions or soluble complexes (without forming precipitates) can be the preferred method when a subsequent step of concentration via evaporation is intended or when in a step subsequent to removal of biomass the fermentation broth is treated so as to promote the formation of complexes that precipitate. Several examples of combinations of compounds that are capable of forming (insoluble) complexes or salts in solution have been described in the art (Table 3). The aforementioned (insoluble) complexes or salts, however, were formed in simple chemical environments that contain almost exclusively the components parts of the (insoluble) complexes formed in solution. In the examples outlined below it is shown that under appropriate experimental conditions some of the (insoluble) complexes or salts can also be formed and isolated from complex fermentation broths.

In another preferred method of the invention the fermentation broth is concentrated by membranes (e.g. semi-permeable micro-sieve membranes that were also used in Examples 1, 3, 4, 5, 6) or by evaporation in order that the precipitation is enhanced. In these methods of the invention, the first and second fermentation product may be produced by one and the same organism. Alternatively, they may be produced by more than one organism. For instance, the first fermentation product may be produced by a first organism and the second fermentation product may be produced by a second organism. In such instances the first and second organisms may be cultured in one fermenter or each organism may be cultured in two separate fermenters whereby the two fermenters are connected through connecting means such as e.g. two pipes or tubings each of which is equipped with at least one micro-sieve. In this fermenter configuration the soluble medium components are circulated between the first and second fermenters through the connecting means comprising the micro-sieve while the micro-sieve prevents circulation of the production organisms. A micro-sieve is herein thus understood to be a device that allows free passage to metabolites and solutes but not to cells. The micro-sieve will thus usually be a (ultra)filter or membrane with a cut-off limit of at least 1.0, 2.0, 5.0, 10.0, 20.0, 100, 200, 500 or 1000 kDa or a pore size/diameter equal to or less than 0.15, 0.22, 0.45 or 0.5 micrometer. Suitable ultrafilters and membranes are well know to the skilled person and are widely available.

Example 1 describes an application of the micro-sieve membranes that may be generalised in the context of the invention. In Example 1 two different micro-organisms are grown in two fermenters running in parallel and connected through two pipes or tubings each of which are equipped with at least one of such micro-sieves. This construction allows the fermentation of different organisms in the two (or more) fermenters in which only the medium is circulated, but the organisms are kept separated. Nonetheless these micro-organisms are capable to exchange metabolites and are thus allowed to communicate without being in direct contact. In Example 1, for instance, one of the two organisms tends to lower the medium pH by excretion of acidic metabolites, whereas the other organism tends to increase the medium pH. Normally, these shifts in pH are prevented by the addition of bases such as NaOH or acids such as H₂SO₄ or HCl. Such pH control can be omitted by the circulating medium in Example 1. Optionally, the use of these micro-sieve membranes mounted together with valves at both ends of the two connecting tubes or pipes between two (or more) fermenters running in parallel may create a semi-separate reactor volume in which the formation of acid/base (insoluble) complexes or salts can be promoted with helper molecules such as metal ions (Me²⁺, e.g. Ca²⁺), ethanol or any other molecules that promote the formation of (insoluble) complexes or salts. The selectivity can be adjusted by the choice of the proper micro-sieve membrane type. Furthermore, one of the connecting tubes or pipes can be equipped with an expansion or sedimentation vessel to which the helper molecules can be added via an additional inlet and (insoluble) complexes or salts formed can be removed via an additional outlet, thus permitting constant removal of products during fermentation. The creation of such additional dead volume in the fermenter system is especially suitable for anaerobic or micro-aerobic processes. The skilled person will appreciate that if one of the two fermenters is replaced by another connective tube or pipe, the micro-sieve/precipitation vessel construction can also be operated as a shunt to the other fermenter. Thus in further embodiments of the methods of invention the connecting means further connect to a vessel that is separated from the first and second fermenters by the micro-sieve. Preferably, in the vessel a condition is applied that promote the formation of an insoluble complex or an insoluble salt between the first and second fermentation products. Preferably, the condition that promotes the formation of an insoluble complex or an insoluble salt between the first and second fermentation products is one or more conditions selected from:

a) a temperature that is lower than the temperature in the first and second fermenters;

b) a pH that differs from the pH in the first and second fermenters;

c) a concentration of a helper molecule that promotes the formation of an insoluble complex or insoluble salt between the first and second fermentation products. A preferred helper molecules is a metal ion or a water-miscible organic solvent. A preferred water miscible organic solvent is a solvent that is miscible with water in any ratio; and,

d) increasing the concentration of the first and second fermentation product by e.g. evaporation (of the fermentation broth, preferably subsequent to removal of cells/biomass).

Thus preferably in the methods of the invention, one of the fermentation products, the first or the second product, is a weak organic acid, a salt thereof or a derivative thereof. A weak organic acid is herein defined as an organic acid with a pKₐ value that higher than 2.6, 2.8, 3.0, 3.2, 3.4, 3.6, 3.8, 4.0, 4.2, 4.4, 4.6 or 4.8. Preferably, the weak acidic compound is selected from the group consisting of organic acids, antibiotics, proteins having an acidic pI, amino acids like aspartic acid or glutamic acid. Preferably, the weak organic acid is selected from the group consisting of organic acids listed above, including salts and derivatives thereof. At the same time in the methods of the invention, the other fermentation product, i.e. the second or the first product, is a weak alkaline compound, a salt thereof or a derivative thereof. A weak alkaline compound is herein defined as a compound with a pK_{b} value that is less than 11, 10.8, 10.6, 10.4, 10.2, 10.0, 9.8, 9.6, 9.4, 9.2, 9.0, 8.8 or 8.6. The pK_{b} value is herein defined as −log₁₀K_{b}, whereby K_{b} is defined as [BH⁺]*[OH⁻] divided by [B]. Preferably, the weak alkaline compound is selected from the group consisting of lysine, arginine, histidine, a polypeptide having an alkaline pI, various antibiotic molecules, alkanediamines.

Preferably in the methods of the invention, at least a part of the first metabolic pathway, the second metabolic pathway or both the first and second metabolic pathways are not naturally present or active in the organism. Preferably, the first and/or second metabolic pathways are introduced or activated in the organism by a genetic modification of the organism. A wide variety of techniques is available to the skilled person for genetic modification of the production organism so as to introduce or activate metabolic pathways. The techniques include classical genetic techniques such as crossing, mating, (inter- and intra-species) protoplast fusion, and mutagenesis by chemicals (e.g. EMS, MMS), by radiation (UV, beta- or gamma radiation), as well as more modern mutagenesis techniques by insertion, gene disruption, gene replacement, transposons, antisense RNA, co-suppression, quelling or RNAi. Mutagenesis may optionally be followed by selection of mutants with the desired modification, e.g. using high throughput screening. Genetic modifications may also be effected by recombinant DNA techniques including e.g. cloning of genes or cDNAs, construction of nucleic acid constructs for expression or inactivation of genes, and transformation, transduction, transfection, infection of the organisms with the nucleic acid constructs, optionally followed by selection of transformants with the desired modification as described above. The skilled person will appreciate that any of the aforementioned techniques may be combined and/or repeated one or more times in order to effect the desired genetic modifications. The examples herein provide references for description of these genetic techniques.

The entire genome sequence of B. subtilis subsp. subtilis strain 168 (taxonomy ID: 224308) has been published (Kunst et al. 1997; http://www.ncbi.nlm.nih.gov/Taxonomy/Browser/wwwtax.cgi?id=224308), whereas more recently the entire genome sequence of C. glutamicum (and of two other Corynebacterium spp.) has been determined (Ikeda and Nakagawa, 2003; Kalinowski et al., 2003), analysed via different Genomics techniques (Kromer et al. (2004) and the relevance for the production of targeted mutants has been assessed (Ohnishi et al., 2002). With a combination of the well established protoplast fusion technique and the more recent Genomics (collective noun for all-omics approaches), including e.g. Comparative or Functional Genomics, Transcriptomics, Proteomics, Metabolomics, Fluxomics and others, a totally new activity has been designed herein, collectively entitled: “GenoMix”. This term is used herein for the combination of all established Genomics technologies normally employed for single species or cultures, but which are also applicable to protoplast fusion products. For example, ProteoMix means the 2D protein and Software and Maldi-TOF-assisted collective analysis of hybrid protein spots or patterns. Similarly, TranscriptoMix means the analysis of integral transcription profiles of fusants and FluxoMix means the analysis of combined metabolite fluxes of either fusion partner.

In the methods of the invention, the production organism may be any organism the cells of which may be cultured in vitro in e.g. a fermenter. Cells from higher organisms like plants and vertebrates, in particular insect and mammalian cells, that may be cultured in vitro are included as production organism for use in the methods of the invention. A preferred production organism is however a microorganism such as a bacterium, fungus, yeast, alga or protozoan. Preferred bacteria for use as production organism include e.g. Acinetobacter sp, Bacillus cereus, Bacillus subtilis, Bacillus thuringiensis, Brevibacterium lactofermentum, Clostridium acetobutylicum, Clostridium perfringens, Corynebacterium sp., Corynebacterium efficiens, Corynebacterium glutamicum, Escherichia coli, Gluconobacter sp, Gluconobacter oxydans, Gluconobacter suboxydans, Lactobacillus plantarum, Lactococcus cremoris, Lactococcus lactis, Methanococcus sp., Pseudomonas sp., Pseudomonas oleovorans, Pseudomonas putida, Pyrococcus furiosus, Ralstonia sp., Rhizobium sp., Sinorhizobium meliloti, Streptococcus sp. and Xanthomonas sp. Preferred filamentous fungi for use as production organism include e.g. Aspergillus sp., Aspergillis nidulans, Aspergillus niger, Aspergillus oryzae, Penicillium sp, Penicillium chrysogenum, Rhizopus oryzae, Trichoderma sp., Trichoderma harzianum, Trichoderma viride and Mortierella alpina. Preferred yeasts for use as production organism include e.g. Saccharomyces sp., Saccharomyces cerevisiae, Pichia pastoris, Pichia sp., Kluyveromyces sp., Hansenula polymorpha and Cryptococcus curvatus. Preferred algae for use as production organism include e.g. Chlorella sp., Chlorella prothecoides, Crypthecodinium sp., Crypthecodinium cohnii, Dunaliella sp., Galdieria sulphuraria, Morinella marinus, Photobacterium profundum, Schizochitrium sp., Shewanella sp., and Traustochytrium sp.

In the methods of the invention, the cells of production organism are preferably cultured or used as viable, preferably intact cells, as opposed to e.g. solvent permeabilised cells or enzyme systems that are isolated and/or reconstituted from cells. A viable cell is herein defined as a cell that has the ability to replicate.

A preferred organism for the production of lysine as first fermentation product and succinate as second fermentation product is Corynebacterium glutamicum. A preferred organism for the production of lysine as first fermentation product and glutamate as second fermentation product is Brevibacterium lactofermentum. A preferred organism for the production of glycerol as first fermentation product and glutamate as second fermentation product is Saccharomyces cerevisiae. A preferred organism for the production of alanine as first fermentation product and citrate as second fermentation product is Aspergillus niger. A preferred organism for the production of fatty acids as first fermentation product and oxalate or acetate as second fermentation product is Cryptococcus curvatus. A preferred organism for the production of a beta-lactam antibiotic, such as penicillin G or V, as first fermentation product and oxalate and/or citric acid as second fermentation product is a fungus of the genus Penicillium.

In yet another aspect the invention relates to a fermentation system comprising at least two different fermentation volumes, wherein each fermentation volume comprises at least one fermenter, wherein the different fermentation volumes are connected through connecting means comprising a micro-sieve that allows circulation of soluble medium components between the different fermenter volumes while preventing circulation of cells of the production organism between the fermentation volumes. Preferably in the fermentation system, wherein the connecting means further connect to a vessel that is separated from the different fermenter volumes by the micro-sieve. The vessel may be used for collection, concentration and/or sedimentation of fermentation products as described above. Further embodiments of the fermentation system are described above, where the methods in which the system is used are set out.

Some advantages of the methods of the invention as compared to conventional aerobic fermentation processes include:
Enhanced substrate efficiency due to compensation of reduction equivalents during balanced production rates of the first and second fermentation product component.
Lower running and investment costs as a result of reduced oxygen demand and heat production thereby avoiding the need for strong aeration and large cooling capacity. In addition, the volumetric productivity can be increased, thus decreasing the contribution of investment costs on final product prices. By combining biosynthetic pathways, either by co-cultivation or co-fermentation of different organisms or by metabolic engineering within one organism in one single fermenter two or more fermentation products can now be produced in single fermentation runs (Examples 2, 3, 4, 5, 6).

Some advantages of the method of invention as compared to conventional anaerobic fermentation processes include:
If the formation of first and second product does not result in complete redox balance respiration will remove surplus reducing power
If the formation of the first and second product does not generate metabolic energy respiration can convert reducing power into metabolic energy
   Both advantages result in a wider product spectrum.

During the fermentative production of an acidic product like citric acid, neutralisation is required to avoid excessive acidification of the fermentation broth. The same is true for any basic final product like lysine. By the simultaneous fermentative production of an acidic (e.g. gluconic acid) and a basic product (e.g. lysine) provide a number of additional advantages:
There is less or no need for the addition of acid or basic pH-controlling compounds, possibly also resulting in higher production rates and/or concentrations
Molecules comprising amino groups and acidic groups may associate to form a(n) (insoluble) complex or salt with reduced solubility that may precipitate. Thus, the (soluble) product concentration in the fermentation broth is reduced, thereby avoiding product feedback inhibition and allowing higher volumetric production rates.
During downstream processing the low solubility of the acid/base (insoluble) complex or salt may be advantageous, because the two complexed components can be purified from much smaller volumes using techniques that require less energy and helper compounds than those in conventional processes from diluted process streams.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### Example 1

### Co-Fermentation of Lysine-Producing Corynebacterium glutamicum and Gluconate-Producing Gluconobacter oxydans Mutants

This example demonstrates the simultaneous production of lysine and gluconic acid by a procedure, denoted co-fermentation and involving cultivation in two fermenters of two bacterial species C. glutamicum and G. oxydans. In order to circumvent the necessity of pH control two fermenters (A for Cg, B for Go) are used, in one of which a basic amino acid is produced whereas in the other fermenter an organic acid is produced. The two fermenters are connected via tubes or pipes that comprise semi-permeable micro-sieves (also mentioned in the general description) that do not permit passage of cells, but only of the medium and all of its components. Thus, this method resembles co-cultivation or co-immobilisation (Aiguo and Peiji, 1998), but in this case without mixing the bacterial species.

C. glutamicum is well known because of its lysine production (Ikeda, 2003). A mathematical model has been designed describing the production process and options for improvements (Ensari and Lim, 2003). In addition, considerable amounts of glutamic acid can be formed (Wijayarathna et al., 2001) and a number of other amino acids. A gluconate overproducing strain of G. oxydans was used in co-fermentation experiments (gluconate production and excretion) together with C. glutamicum (lysine production and excretion). C. glutamicum is facultative anaerobic, whereas G. oxydans is obligate aerobic.

During overproduction of gluconate and lysine, consumption of excreted products (metabolites) by the production organism or its co-fermentation partner should be prevented (see e.g. Lee et al., 1998). Therefore, gluconate and lysine uptake mutants of C. glutamicum and G. oxydans are prepared of either species participating in the co-cultivation procedure (see e.g. Popkin and Maas, 1980; Revelles et al., 2004). Moreover, the gluconate operon (gene cluster) and other genes indirectly involved in gluconate metabolism have been identified in several organisms including, e.g. Bacillus subtilis (Fujita and Fujita, 1987; Fujita and Miwa, 1994) and Escherichia coli (Izu et al., 1997). Additional information from http://www.ncbi.nlm.nih.gov/entrez/ allows us to identify, isolate and to disrupt genes in both C. glutamicum and G. oxydans encoding gluconate-degrading enzymes, more specifically the gluconokinase gene, involved in the conversion of D-gluconate to D-6-P-gluconate. Because there is no known lysine uptake mutant available of G. oxydans, a number of candidate strains are isolated following conventional UV mutagenesis. For selection minimal medium is employed supplemented with essential components and lysine as sole carbon and energy source. Colonies not surviving after subsequent replica-plating are further analysed. Although this method allows the occasional isolation of proper candidate mutants, the method is laborious especially due to their identification and the generation of lethal mutations not related to lysine uptake. In addition, the accumulation of random non-related mutations is unwanted in industrial production strains. Therefore, also the direct approach using silencing of the lysine transporter (lysI) gene in G. oxydans by homologous recombination is performed. For isolating the lysI gene from G. oxydans information for BLAST searches and PCR primer design was used from the NCBI website http://www.ncbi.nlm.nih.gov/entrez and more specifically from organisms listed in Appendix 1a. Details on vector constructions and transformation procedures are as described by Van der Rest et al. (1999), Jang and Britz (2000), Jang et al. (2001) and Kreutzer et al. for C. glutamicum (Patent US2003/0162269), using strain DSM 5715 as hosts, plasmid pEC7 as the cloning and expression vector and electroporation for DNA transfer (Creaven et al., 1994). For G. oxydans strain ATCC 9937 plasmid pBBR122 (mob Cm^{r} Km^{r}) is used as the shuttle cloning vector, and electroporation as described (Fukaya et al., 1985; Mostafa et al., 2002). In order to promote (homologous) integration into the host genome and to improve transformation efficiencies vectors are linearised by restriction with uniquely cutting restriction enzymes prior to electroporation.

The resulting recombinants are checked by Southern blot analyses, PCR and physiological techniques (growth tests as indicated above).

As indicated in the general description for the current Example the two organisms C. glutamicum and G. oxydans are grown in two fermenters running in parallel and connected via two pipes equipped with semi-permeable micro-sieves and valves at both ends, thus allowing circulation of the medium between the two fermenters without mixing the cells.

For co-fermentation, the two bacterial species are used as inocula for two interconnected 1 L active volume stirred tank reactors (Applikon, Schiedam, The Netherlands) from individual overnight precultures (2*100 mL minimal medium containing glucose and supplements as required; the composition is adjusted to allow growth of both organisms).

During fermentation (experiment A) all essential parameters, such as glucose concentration, dO₂, CO₂ concentration, pH, stirrer speed and biomass are determined as well as concentrations of lysine, and gluconate both in the medium and in extracted cell pellets. As a control (experiment B) the same experiment is repeated with all four valves in the pipes connecting the two fermenters kept closed during the entire fermentation run. As in experiment A the pH is not adjusted and therefore decreased in the G. oxydans run and increased in the C. glutamicum run. Aeration is kept optimal by a stirrer speed of 1000 rpm and an aeration of 1 vvm, because the synthesis of gluconic acid from glucose requires oxygen. Fermentations are terminated after 24 h.

The results indicate that during the co-fermentation (experiment A) the pH only shifts from 7.3 (initial value) to 6.5 at the end of the fermentation run as measured in samples taken from the connective tubes between the two fermenters, whereas in individual fermentation runs (Experiment B) the pH for the G. oxydans culture may drop to pH 3.2 and for C. glutamicum may increase to pH 8.9. In addition, lysine and gluconate productivities are increased probably due to the lack of deleterious pH shifts in the co-fermentation experiment.

### Example 2

### Intergeneric Protoplast Fusion of Lysine-Producing Corynebacterium glutamicum and Acetate-Producing Bacillus subtilis; Combination of Lysine and Acetate Synthetic Pathways (GenoMix)

In Bacillus subtilis, the products of the pta and ackA genes, phosphotransacetylase and acetate kinase, play a crucial role in the production of acetate, one of the most abundant by-products of carbon metabolism in this gram-positive bacterium (Presecan et al., 1999).

In order to combine this acetate synthetic pathway of B. subtilis and the lysine synthetic pathway of C. glutamicum for the current example we employ the technique of intergeneric protoplast fusion. As for plants and fungi also for bacteria protoplast fusion techniques have been widely employed in order to combine desired traits of either parent organism or strain. In fact, this is the only artificial recombinant DNA technique (other than conventional DNA transfer techniques) that ensures the presence of all essential DNA sequences in the fusant, at least initially. Although a large number of unpredictable recombination events may be expected to occur during which redundant biosynthetic and regulatory DNA sequences may be eliminated, there are examples of rather stable situations, which can be characterised by intermediate phenotypes of either parent.

The procedure developed worked very well with bacterial strains of G. oxydans (ATCC 9937) and Acetobacter melanogenus (NCIM2259), Erwinia herbicola (ATCC 21998), Pseudomonas chlororaphis (NCIM2041) and Corynebacterium sp. (ATCC 31090) (Verma et al. 1989). Intergeneric protoplast fusion between 2,5-diketo-gluconic acid producing G. oxydans (ATCC 9937) and a mutant strain of Corynebacterium sp. (ATCC 31090), capable of reducing 2,5-diketo-gluconic acid to 2-keto-L-gulonic acid, a penultimate step in vitamin C production, resulted in viable recombinants. Some of the fusion products exhibited the capacity to convert D-glucose to 2-keto-L-gulonic acid, but the conversion rate was low (Verma et al. 1992).

Hybrids between a strain of B. subtilis having the ability to degrade xylan and other complex polysaccharides and C. acetoacidophilum, a lysine producer, were prepared by protoplast fusion (Deb et al. 1990). The results demonstrate that intergeneric gene transfer takes place through protoplast fusion between these 2 important genera of bacteria and some of the fusants inherit the useful traits of both the parents (Deb et al. 1990).

For the current example acetate and lysine uptake mutants are produced by random mutagenesis of lysine-producing C. glutamicum (ATCC 21253) and an acetate producing strain of B. subtilis (ATCC 6633) by selection for their inability to grow on acetate and lysine as sole carbon and energy source, respectively. There is ample evidence that C. glutamicum is able to use acetate as a single or as a co-substrate with glucose, but until now the gene for the postulated combined acetate/propionate transporter has not be identified in the C. glutamicum entire genome sequence (Gerstmeir et al., 2003). Therefore, we overexpress the ramB gene, a negative transcriptional regulator of genes involved in acetate metabolism of C. glutamicum (Gerstmeir, 2004). Similarly, In B. subtilis the gene for lysine uptake and excretion, unlike lysI in G. oxydans (used in Example 1), has not been annotated in the entire genome sequence (Kunst et al., 1997) and appears to be part of a more general amino acid Ca²⁺/H⁺ antiporter (Matsushita et al., 1986).

The new mutant strains, C. glutamicum ATCC 21253 acu⁻ (acetate uptake negative) and B. subtilis ATCC 6633 lyu⁻ (lysine uptake negative) are used for fusion experiments according to methods adopted from Deb et al. (1990) and Verma et al. (1992). Subsequently, the current example employs additional rounds of UV mutagenesis to delete redundant DNA sequences and/or enzyme activities and to select for best lysine and acetate-producing bacterial fusant strains in high throughput screening programs and GenoMix technologies.

The stability of fusants as a function of growth and the number of generations was investigated using several criteria, as follows: 1—morphological characteristics reminiscent of the parental phenotypes; 2—presence of selected specific DNA sequences using primer sequences that recognised published DNA sequences and/or enzyme activities of both fusing partners; 3—full genomic AFLP analysis, randomly showing the presence or possible disappearance of DNA sequences; 4—continued capability to produce lysine and acetate.

The best performing strain CBAF03-55 is subjected to fermentation experiments as described for C. glutamicum.

The results indicate that the fusant strain is capable of producing both lysine and acetate, whereas in C. glutamicum ATCC 21253 ace⁻ only traces of acetic acid can be detected. By fusing industrial production organisms and subsequent mutagenesis treatment novel fusant strains can be obtained that exhibit high productivities for both of the main basic and acidic products of the parental strains. Thus, by combining two different single-product fermentation processes into one new process involving the production of a first and a second fermentation product the volumetric productivity can be increased.

### Example 3

### Simultaneous Production of Lysine and Glutamate by Brevibacterium lactofermentum

Various coryneform bacteria are able to produce amino acids, such as glutamate and lysine. The conversion of glucose into glutamate generates reducing power, the conversion of glucose into lysine requires reducing power. Simultaneous production of both amino acids by a microorganism should therefore lead to a high conversion efficiency and low oxygen demand.

It has been described for B. lactofermentum that glutamate and lysine can be produced simultaneously by (Shiratsuchi et al., 1995; EP0780477 al, example 6), but this did not result in a higher yield compared to the situation in which only lysine was formed. We are able to increase the efficiency of the process by applying an oxygen limitation to a culture in which both amino acids were produced simultaneously. Furthermore, it was suggested that the L-lysine and L-glutamic acid produced would exist in solution as L-lysine.L-glutamate molecules or complexes.

Lysine and glutamate are produced in a fermentation by B. lactofermentum AJ12993 essentially as described (Shiratsuchi et al., 1995; EP0780477, Example 6), under oxygen excess conditions and under oxygen limitation. Oxygen excess conditions are maintained by a stirrer speed of 1000 rpm and an aeration of 1 vvm. Oxygen limiting conditions are maintained by an aeration of 0.2 vvm and an agitation of 300 rpm. Results presented in Table 1, indicate that by applying an oxygen limitation the product yield increases and the oxygen demand decreases. Furthermore, by using the micro-sieve and sedimentation device (as indicated in the general description), a sediment comprising the lysine.glutamate complex is obtained.

TABLE 1
Maximal product concentrations formed by B. lactofermentum
under oxygen excess and oxygen limitation.

| | Oxygen | Oxygen |
|---|---|---|
| | excess | limitation |
| Glucose (g/L)* | 150 | 153 |
| Lysine (g/L)* | 44 | 46 |
| Glutamate (g/L)* | 18 | 40 |
| Biomass (g/L cell dry weight) | 18 | 14 |
| O₂/C_{glucose} | 0.38 | 0.27 |

*Possible involvement of use of vitamins, antibiotics, yeast extract, etc. as alternative C-source and possible additional products not accounted for.

The lower oxygen demand of the process thus results in higher productivities in industrial processes in which oxygen is rate limiting. Furthermore, the formation of two commercially interesting products in one single fermentation run increases volumetric productivity.

### Example 4

### Simultaneous Production of Citric Acid and Alanine by Aspergillus niger

In order to increase the total product yield by a citric acid-producing A. niger strains MW131 (wild-type control with irrelevant markers cspA1, goxC17) and MW129 (pyrA, uracil-auxotrophic recipient for plasmid DNA) along with the second fermentation product alanine, a set of transformants is generated, that overexpres the Bacillus sphaericus alanine dehydrogenase (alaD) gene (Hols et al., 1999). For this purpose the promoterless 1.22 kb SmaI-HindIII fragment from plasmid pBM20alaD is cloned into a pMTL23-based vector comprising the A. nidulans GPDA promoter and terminator sequences (Punt et al., 1987), yielding plasmid pANala. This vector is introduced into A. niger MW129 by co-transformation with the pyrA gene (Ruijter et al., 1997). From a number of co-transformants that contain varying copies of the pANala vector one strain is selected that demonstrates highest alaD expression levels (strain AnigAF129-11). Parallel fermentation of the wild-type and the recombinant strain is carried out as described by Ruijter et al. (1997, 2000) as follows: mycelium is cultured at 30° C. in a medium optimised for citric acid production containing 0.5 g KH₂PO₄, 2.5 g (NH₄)₂SO₄, 0.25 g MgSO₄.7H₂O, 6.5 mg FeSO₄.7H₂O, 1.3 mg ZnSO₄.7H₂O and 140 g decationised glucose/L using 1 L active volume stirred tank reactors (Applikon, Schiedam, The Netherlands). From the fermentation start oxygen excess conditions are maintained by a stirrer speed of 1000 rpm and an aeration of 1 vvm. After day three (around mid-log phase) oxygen-limiting conditions are applied by an aeration of 0.2 vvm and an agitation of 300 rpm. Fermentations are terminated after 7 days and analyses performed.

The results indicate that the transformant (AnigAF 129-11) produces citric acid and a considerable quantity of alanine with a lower quantity of biomass than the wild-type (NW131). Furthermore, by using the micro-sieve and sedimentation device (as indicated in the general description), a sediment comprising the alanine.citrate complex is obtained.

By overexpression of the gene for alanine biosynthesis into a citric acid producing A. niger strain the total productivity of the first plus the second fermentation products (citric acid and alanine, respectively) thus increases in industrial processes in which oxygen is rate limiting. Furthermore, the formation of two commercially interesting products in one single fermentation run increases volumetric productivity.

### Example 5

### Coordinate Expression of Genes of the Lysine- and Succinate-Synthetic Pathways Pathways in Corynebacterium glutamicum

Succinate is an important intermediate for a large number of derived products, including commodity chemicals, such as formate, maleate, maleimide, 1,4-butanediol, aspartate, itaconate, dimethyl/dyethyl succinate, tetrahydrofuran, gammabutyrolactone, succinimide, maleic anhydride, adipate, polyesters, solvents, and specialty chemicals such as food ingredients, plant growth stimulants, pharmaceutical intermediate, biophosphors, flavor additive, acidulant.

In order to achieve coordinated expression of the genes of the lysine- and succinate biosynthetic pathways by recDNA technology the following approach was chosen: overexpression of the homologous succinate-synthetic genes in a lactate dehydrogenase (ldh)-negative strain of C. glutamicum. Following the approach presented by Yukawa et al. (2003) we knock out the C. glutamicum strain ATCC 31831 lactate dehydrogenase (ldh) gene and subsequently overexpress the homologous genes involved in succinate metabolism, namely the succinate semialdehyde dehydrogenase (ssdh) and succinate dehydrogenase (sdh) subunit genes. The sequences of these genes may be retreived from the C. glutamicum complete genome accession number NC_{—}003450 (Corynebacterium glutamicum ATCC 13032), gi23308765 and isolated following nested PCR. Successful and efficient transformation has been described for C. glutamicum with single and cointegrate integrative transformation vectors (Ikeda and Katsumata, 1998; Kotrba et al., 2003; Kreutzer et al. 2003; Kirchner and Tauch, 2003; Tauch et al., 2003). We employ the system employed by Kreutzer et al. (Patent US2003/0162269), using strain DSM5715 as a host, plasmid pEC7 as the cloning vector that allows multiple inserts and electroporation for DNA transfer. A large number of recombinant strains thus obtained is isolated and analysed. The best performing strain (CAF04-26; ldh, ssdh, sdh) and the control strain ATCC 31831 are subjected to parallel fermentation experiments and product analyses.

The results indicate that the novel recombinant strain produces higher concentrations of both lysine and succinate as compared to the control strain. Furthermore, by using the micro-sieve and sedimentation device (as indicated in the general description), a sediment comprising the lysine.succinate complex is obtained as described by Prasad and Vijayan (1991).

Overexpression of relevant genes for the coordinate biosynthesis of a reduced amino acid such as lysine (first fermentation product) and a more oxidised organic acid such as succinic acid (second fermentation product) thus results in higher productivities of the first and the second fermentation products in industrial production processes. Furthermore, the formation of two commercially interesting products in one single fermentation run increases volumetric productivity.

### Example 6

### Coupling the Penicillin- and Oxalate Production Pathways in Penicillium chrysogenum

The current Example exploits the coupling of penicillin and oxalate biosynthetic pathways. Incomplete oxidation of glucose to oxalic acid during oxygen-limited and nitrate-dependent growth yields reducing power for members of the β-lactam antibiotics family. After excretion, oxalic acid is complexed to Ca-ions, leading to the formation of non-soluble Ca-oxalate crystals and higher penicillin yields.

The industrial production of beta-lactam antibiotics by fermentation over the past 50 years is one of the outstanding examples of biotechnology. Today, the beta-lactam antibiotics, particularly penicillins and cephalosporins, represent the world's major biotechnology products with worldwide dosage form sales of approximately 15 billion US dollars or approximately 65% of the total world market for antibiotics. Over the past five decades, major improvements in the productivity of the producer organisms, P. chrysogenum and Acremonium chrysogenum (syn. Cephalosporium acremonium) and improved fermentation technology have culminated in enhanced productivity and substantial cost reduction. Major fermentation producers are now estimated to record harvest titers of 40-50 g/L for penicillin and 20-25 g/L for cephalosporin C. Recovery yields for penicillin G or penicillin V are now >90%. Chemical and enzymatic hydrolysis process technology for 6-aminopenicillanic acid or 7-aminocephalosporanic acid is also highly efficient (approximately 80-90%) with new enzyme technology leading to major cost reductions over the past decade (Elander, 2003). This implies that additional process improvements can mainly be expected from further strain development and fermentation process optimisation as described herein.

Several fungal species, including industrial organisms, like Penicillium spp. and Aspergillus spp. are well known for their capacity to efficiently produce a number of organic acids including oxalic acid (Cunningham and Kuiack, 1992; Carneselle et al., 1998) and a number of the biosynthetic genes have been identified, also in other fungi (see: http://www.ncbi.nlm.nih.gov/entrez/; Balmforth and Thomson, 1984; Mehta and Datta, 1991; Ruijter et al., 1999; Kesarwani et al., 2000; Pedersen et al., 2000; Azam et al., 2002).

Evidence has been provided for a cytoplasmic pathway of oxalate biosynthesis in A. niger (Kubicek et al., 1988). Liquid cultures of nitrate-grown fungi produce substantial amounts of oxalic acid, whereas in ammonium-containing medium oxalic acid was only detected in small amounts (Gharieb and Gadd, 1999). The external pH appeared to be the main factor governing oxalic acid production by A. niger. A glucose-oxidase-negative mutant produced substantial amounts of oxalic acid as long as the pH of the culture was 3 or higher. The major acidic metabolites produced by P. bilaii in a sucrose nitrate liquid medium were found to be oxalic acid and citric acid. Citric acid production was promoted under nitrogen-limited conditions, while oxalic acid production was promoted under carbon-limited conditions. Citric acid was produced in both growth and stationary phases, but oxalic acid production occurred only in stationary phase (Cunningham and Kuiack, 1992). However, under the industrial fermentation process conditions employed the oxalate synthesis negatively correlated with penicilline productivity (Diez et al., 1986, Recd. 1987). Therefore, in the current example the production process was adapted based on a detailed stoichiometric model (Van Gulik et al., 2000) for growth and penicillin G production in P. chrysogenum. From an a priori metabolic flux analysis using this model it appeared that penicillin production requires significant changes in fluxes through the primary metabolic pathways. This is brought about by the biosynthesis of carbon precursors for the beta-lactam nucleus and an increased demand for NADPH, mainly for sulfate reduction. Basic tools for P. chrysogenum transformation and cotransformation (both vectors with a variety of dominant selectable markers and transformation protocols) are available (Graessle et al., 1997; Casqueiro et al., 1999; Theilgaard et al., 2001; Bañuelos et al., 2003) and the transgene stability has been assessed in fermentation runs (Renno et al., 1990).

For the current example genes involved in oxalate biosynthesis are identified from a number of organisms (references listed above and http://www.ncbi.nlm.nih.gov/entrez/), which information is used for the isolation of the equivalent genes from P. chrysogenum (and A. niger).

Genes are isolated and employed encoding: 1) Oxalate-CoA transferase (oxalate converted to succinate); 2) Oxalate decarboxylase (oxalate converted to formate); 3)

Glyoxylate dehydrogenase (oxalate synthesised from glyoxylate), 4) (S)-2-Hydroxy-acid oxidase (oxalate synthesised from glyoxylate); 5) Ascorbate 2,3-dioxygenase (oxalate synthesised from ascorbate). Several transformation vector constructs were prepared aiming at overexpression (genes 3, 4, 5) or knocking out (genes 1, 2) the P. chrysogenum equivalents.

We employ the P. chrysogenum Wisconsin 54-1255 pyrG(−) strain, an uracil auxotroph with a frameshift mutation in the pyrG gene (Bafñuelos et al., 2001), as recipient strain in transformation procedures. The autonomously replicating plasmid pAM9L (pyrG) is used for the selection of primary transformants, whereas the integrative plasmid pEF43, that contains the bleomycin/phleomycin-resistance gene (ble) from Streptoalloteichus hindustanus under the control of a fungal promoter (the cefEF gene promoter of Acremonium chrysogenum), is used for secundary dominant selection of strains comprising co-transforming DNA. Co-transforming genes (1-5, listed above) are cloned into vector pEF43 comprising the endogenous gene-specific promoter and terminator sequences for overexpression (genes 3, 4, 5) or 5′- and 3′-deletion mutants only comprising the internal fragments of the structural genes (1, 2, each retaining about 750-1000 bps) for gene silencing. In order to promote (homologous) integration into the host genome and silencing of genes 1 and 2, corresponding vectors are linearised with restriction enzymes (Casqueiro et al., 1999) uniquely cutting within the homologous P. chrysogenum sequences prior to electroporation (Van de Rhee et al. 1996).

Plasmids pAML9L and the five pEF43-derivatives are mixed together (100 ng of each) in shotgun co-transformation experiments using electroporation. 5-Fluoro-orotic acid (FOA), uridine and phleomycin are added to Czapek medium at a final concentration of 1 mg/ml, 100 and 30 μg/ml, respectively as required (Díez et al., 1987).

A few multiple transgenics are identified that contain co-integrates of the donor DNA sequences are grown in 96-microwell plates and analysed by RT-PCR for the presence and relative expression of genes 1-5 and for the presence of penicilline and oxalic acid. The best performing strain (PAF03-247) in microwell plates is further subjected to fermentation experiments in 1 L active volume stirred tank reactors (Applikon, Schiedam, The Netherlands) with medium composition (in this example with 100 g/L glucose) and preparation and chemostat system and cultures essentially as described by van Gullik et al. (2000).

During fermentation and after the aeration-enhanced growth phase Ca²⁺, Co²⁺ and Zn²⁺ (Me²⁺) ions are tested for their ability to efficiently bind oxalate and other acid ions in the fermentation broth and, thus, drive the reactions into the formation of final products Me-oxalate and penicillin.

Essential results for strain PAF03-247 and control strain PAF03-9 that exclusively contain the pyrg gene from pAML9L demonstrate that by the introduction of (additional) oxalate synthetic capacity and by inhibition of oxalate breakdown or turnover, aided by extracellular precipitation as Me- or Ca-oxalate the total productivity of both penicillin and oxalate increase. Furthermore, by using the micro-sieve and sedimentation device (as indicated in the general description), a calcium-oxalate sediment is obtained.

### Example 7

### Coupling of Poly-Unsaturated Fatty Acid (PUFA) and Oxalic/Acetic Acid Production in the Oleaginous Yeast Cryptococcus curvatus

The human consumption of PUFAs has been associated with health-promoting effects (e.g. prevention of coronary diseases and intestinal cancer) and infant brain development.

In order to increase the productivity of PUFAs by the oleaginous yeast C. curvatus, which belongs to the group of basidiomycetes (to which for instance also the common white button mushroom Agaricus bisporus belongs), coordinate production and consumption of reducing equivalents is achieved by fermentation process engineering. It has been suggested (Ratledge, 2002) that in lipid accumulating fungi such as Mucor circinelloides and Mortierella alpina the key step limiting fatty acid biosynthesis is the provision of sufficient reducing equivalents. There are reports on oxalic or formic acid production and excretion in other basidiomycetes, but not yet in C. curvatus. Therefore, we set out to introduce additional capacity into the fermentation process of C. curvatus to overproduce and excrete the incomplete oxidation products oxalic and acetic acid. The oxalate/acetate mixture is subsequently removed from the fermentation broth using CaCO₃, thus forming Ca-oxalate/acetate crystals as a sink.

Strain C. curvatus ATCC 20509 is subjected to parallel fermentation experiments (Meesters et al., 1996) including an oxygen depletion step at the mid-log phase in fermentor A and a control fermentation run B, in which the oxygen supply is kept optimal.

From the fermentation start oxygen excess conditions are maintained by a stirrer speed of 1000 rpm and an aeration of 1 vvm in both fermentor A and B. After 24 h (around mid-log phase) oxygen-limiting conditions are applied only to fermentor A by an aeration of 0.2 vvm and an agitation of 300 rpm. Fermentations are terminated after 72 h and analyses performed.

As a result we observe that the generation of additional reducing equivalents indeed promotes fatty acid biosynthesis and accumulation. The micro-sieve/sedimentation device that is used as a shunt to fermenter A allowed the precipitation and separation of Ca-oxalate crystals.

The results indicate that lowering the oxygen transfer, subsequent oxalate and acetate synthesis and the extracellular removal of oxalate and acetate promotes the productivity of industrial processes aiming at the production of (long chain poly-unsaturated) fatty acids in oil-producing micro-organisms.

### Example 8

### Formation of Non-Ideal Solutions, Salts or (Insoluble) Complexes Between Lysine and Lactic Acid

Formation of couples of two organic counterions in cells has advantages. Firstly, the cell does not experience a high concentration since the formation of couples reduces the concentration of the single ions. Therefore, there will be less feedback inhibition. Secondly, downstream processing in smaller volumes of the fermentation medium will be possible when precipitation or complex formation occurs. Many salts consisting of two organic counterions have been described in the scientific literature (Table 3). In the current experiment, couples of lysine (L-lysine.HCl is used) with lactic acid, acetic acid, succinic acid or citric acid are tested on their capacity for complex formation. In this experiment, the conductivity of solutions with two counterions is measured. The counterions are mixed in equimolar ratios, at concentrations ranging from 0 to 2 M. By using dilutions from stock solutions in 2 M KOH, the pH is set at 7 at all concentrations used for conductivity measurements.

Table 2 shows that the conductivity of a solution with L-lysine and lactic acid reaches a maximum at concentrations between 1.0 and 1.2 M and then decreases until the highest concentration used (2.0 M). This implies that interactions (non-ideal solutions, complexation, crystallisation) occur that mimic lower concentrations of the components. Thus, in an in vivo situation cells will experience lower concentrations of the components whereas their actual concentration may be much higher (even >2 M).

TABLE 2
Conductivities of equimolar solutions of lysine and lactic acid.

| Concentration | Conductivity |
|---|---|
| (M) | (mS/cm) |
| 0 | 0.0005 |
| 0.2 | 25.3 |
| 0.4 | 43.1 |
| 0.6 | 55.4 |
| 0.8 | 63.6 |
| 1.0 | 68.2 |
| 1.2 | 68.8 |
| 1.4 | 67.8 |
| 1.6 | 63.2 |
| 1.8 | 56.3 |
| 2.0 | 48.9 |

Similar results are obtained for couples of lysine and acetic acid, lysine and succinic acid and lysine and citric acid.

Also complexes can be formed between larger molecules, like proteins, and short carboxylic acids, such as κ-casein. lactate (Oeda, 1978).

### REFERENCES

1. Aiguo J, Peiji G. Synthesis of 2-keto-L-gulonic acid from gluconic acid by co-immobilized Gluconobacter oxydans and Corynebacterium sp. Biotechnology Letters 20 (1998) 939-942.
2. Azam M, Kesarwani M, Chakraborty S, Natarajan K, Datta A. Cloning and characterization of the 5′-flanking region of the oxalate decarboxylase gene from Flammulina velutipes. Biochem J. 367 (2002) 67-75.
3. Balmforth A J, Thomson A. Isolation and characterization of glyoxylate dehydrogenase from the fungus Sclerotium rolfsii. Biochem J 218 (1984) 113-118.
4. Bañuelos O, Casqueiro J, Gutiérrez S, Martín J F. Intrachromosomal recombination after targeted monocopy integration in Penicillium chrysogenum: stabilization of the direct repeats to prevent loss of the inserted gene. Curr Genet. 39 (2001) 231-236.
5. Bañuelos O, Naranjo L, Casqueiro J, Gutierrez S, Martin J F. Co-transformation with autonomous replicating and integrative plasmids in Penicillium chrysogenum is highly efficient and leads in some cases to rescue of the intact integrative plasmid. Fungal Genet Biol 40 (2003) 83-92.
6. Carneselle C, Bohlmann J T, Nunez M J, Lema J M. Oxalic acid production by Aspergillus niger. Bioprocess Engineering 19 (1998) 247-252.
7. Casqueiro J, Gutierrez S, Bañuelos O, Hijarrubia M J, Martin J F. Gene targeting in Penicillium chrysogenum: disruption of the lys2 gene leads to penicillin overproduction. J Bacteriol (1999) 1181-1188.
8. Creaven M, Fitzgerald R, O′-Gara F. Transformation of Gluconobacter oxydans subsp. suboxydans by electroporation. Canadian Journal of Microbiology 40 (1994) 491-494.
9. Cunningham J E, Kuiack C. Production of citric and oxalic acids and solubilization of calcium phosphate by Penicillium bilaii. Applied and Environmental Microbiology 58 (1992) 1451-1458.
10. Deb J K, Malik S, Ghosh V K, Mathai S, Sethi R. Intergeneric protoplast fusion between xylanase producing Bacillus subtilis LYT and Corynebacterium acetoacidophilum ATCC 21476. FEMS Microbiol Lett. 59 (1990) 287-292.
11. Deppenmeier U, Hoffmeister M, Prust C. Biochemistry and biotechnological applications of Gluconobacter strains. Appl Microbiol Biotechnol 60 (2002) 233-242.
12. Diez A M F, Abrahao N J, Cunha B C D A. Oxalic acid in penicillin industrial fermentation. Revista de Microbiologia 17 (1986, Recd. 1987) 364-370.
13. Díez B, Alvarez E, Cantoral J M, Barredo J L, Martin J F. Selection and characterization of pyrG mutants of Penicillium chrysogenum lacking orotidine-5′-phosphate decarboxylase and complementation by the pyr4 gene of Neurospora crassa. Curr Genet. 12 (1987) 277-282.
14. Elander R P. Industrial production of beta-lactam antibiotics. Appl Microbiol Biotechnol 61 (2003) 385-392.
15. Ensari S, Lim H C. Kinetics of L-lysine fermentation: a continuous culture model incorporating oxygen uptake rate. Applied Microbiology and Biotechnology 62 (2003) 35-40.
16. Felder M, Gupta A, Verma V, Kumar A, Qazi G N, Cullum J. The pyrroloquinoline quinone synthesis genes of Gluconobacter oxydans. FEMS Microbiology Letters 193 (2000) 231-236.
17. Fujita Y, Fujita T. The gluconate operon gnt of Bacillus subtilis encodes its own transcriptional negative regulator. Proceedings of the National Academy USA 13 (1987) 4524-4528.
18. Fujita Y, Miwa Y. Catabolite repression of the Bacillus subtilis gnt operon mediated by the CcpA protein. Journal of Bacteriology 176 (1994) 511-513.
19. Fukaya M, Okumura H, Masai H, Uozumi T, Beppu T. Development of A host-vector system for Gluconobacter suboxydans. Agricultural and Biological Chemistry 49 (1985) 2407-2412.
20. Gerstmeir R, Wendisch V F, Schnicke S, Ruan H, Farwick M, Reinscheid D, Eikmanns B J. Acetate metabolism and its regulation in Corynebacterium glutamicum. J Biotechnol. 2003 Sep. 4; 104(1-3):99-122.
21. Gerstmeir R, Cramer A, Dangel P, Schaffer S, Eikmanns B J. RamB, a novel transcriptional regulator of genes involved in acetate metabolism of Corynebacterium glutamicum. J. Bacteriol. 186 (2004) 2798-809.
22. Gharieb M M, Gadd G M. Influence of nitrogen source on the solubilization of natural gypsum (CaSO4.2H₂O) and the formation of calcium oxalate by different oxalic and citric acid-producing fungi. Mycological Research 103 (1999) 473-481.
23. Graessle S, Haas H, Friedlin E, Kurnsteiner H, Stoffler G, Redl B. Regulated system for heterologous gene expression in Penicillium chrysogenum. Appl Environ Microbiol 63 (1997) 753-756.
24. Hols P, Kleerebezem M, Schanck A N, Ferain T, Hugenholtz J, Delcour J, de Vos W M. Conversion of Lactococcus lactis from homolactic to homoalanine fermentation through metabolic engineering. Nat. Biotechnol. 17 (1999) 588-592.
25. Ikeda M. Amino acid production processes. Adv Biochem Eng Biotechnol 79 (2003) 1-35. Review.
26. Ikeda M, Katsumata R. A novel system with positive selection for the chromosomal integration of replicative plasmid DNA in Corynebacterium glutamicum. Microbiology 144 (1998) 1863-1868.
27. Ikeda M, Nakagawa S. The Corynebacterium glutamicum genome: features and impacts on biotechnological processes. Appl Microbiol Biotechnol 62 (2003) 99-109. Review.
28. Isturiz T, Celaya J. The metabolism of gluconate in Escherichia coli. The subsidiary system and the nature of the gntS gene. Journal of Basic Microbiology 37 (1997) 105-114.
29. Izu H, Adachi O, Yamada M. Purification and characterization of the Escherichia coli thermoresistant gluconokinase encoded by the gntK gene. FEBS Letters 394 (1996) 14-16.
30. Izu H, Adachi O, Yamada M. Gene organization and transcriptional regulation of the gntRKU operon involved in gluconate uptake and catabolism of Escherichia coli. Journal of Molecular Biology 267 (1997) 778-793.
31. Jang K H, Britz M L. Improved electrotransformation frequencies of Corynebacterium glutamicum using cell-surface mutants. Biotechnology Letters 22 (2000) 539-545.
32. Jang K H, Chambers P J, Chun U H, Britz M L. Characterization of the cell-surface barriers to plasmid transformation in Corynebacterium glutamicum. Journal of Microbiology and Biotechnology 11 (2001) 294-301.
33. Kalinowski, J., Bathe, B., Bartels, D., Bischoff, N., Bott, M., Burkovski, A., Dusch, N., Eggeling, L., Eikmanns, B. J., Gaigalat, L., Goesmann, A., Hartmann, M., Huthmacher, K., Kramer, R., Linke, B., McHardy, A. C., Meyer, F., Mockel, B., Pfefferle, W., Puhler, A., Rey, D. A., Ruckert, C., Rupp, O., Sahm, H., Wendisch, V. F., Wiegrabe, I., and Tauch, A. The complete Corynebacterium glutamicum ATCC 13032 genome sequence and its impact on the production of L-aspartate-derived amino acids and vitamins. J Biotechnol 104 (2003) 5-25.
34. Kanagasundaram V, Scopes R. Isolation and characterization of the gene encoding gluconolactonase from Zymomonas mobilis. Biochim Biophys Acta 29 (1992) 198-200.
35. Kesarwani M, Azam M, Natarajan K, Mehta A, Datta A. Oxalate decarboxylase from Collybia velutipes. Molecular cloning and its overexpression to confer resistance to fungal infection in transgenic tobacco and tomato. J Biol Chem 275 (2000) 7230-7238; Erratum in: J Biol Chem 275 (2000) 15600.
36. Kirchner O, Tauch A. Tools for genetic engineering in the amino acid-producing bacterium Corynebacterium glutamicum. Journal of Biotechnology 104 (2003) 287-299.
37. Kotrba P, Inui M, Yukawa H. A single V317A or V317M substitution in enzyme II or newly identified β-glucoside phosphotransferase and utilization system of Corynebacterium glutamicum R extends its specificity towards cellobiose. Microbiology 149 (2003) 1569-1580.
38. Kreutzer C, Hans S, Rieping M, Mockel B, Eggeling L, Sahm H, Patek M. L-lysine-producing Corynebacteria and process for the preparation of L-lysine. Patent US2003/0162269.
39. Kromer J O, Sorgenfrei O, Klopprogge K, Heinzle E, Wittmann C. In-depth profiling of lysine-producing Corynebacterium glutamicum by combined analysis of the transcriptome, metabolome, and fluxome. J. Bacteriol. 186 (2004) 1769-1784.
40. Kronemeyer W, Peekhaus N, Kramer R, Sahm H, Eggeling L. Structure of the gluABCD cluster encoding the glutamate uptake system of Corynebacterium glutamicum. J Bacteriol 177 (1995) 1152-1158.
41. Kubicek C P, Schreferl K G, Wohrer W, Rohr M. Evidence for a cytoplasmic pathway of oxalate biosynthesis in Aspergillus niger. Applied and Environmental Microbiology 54 (1988) 633-637.
42. Kunst F, Ogasawara N, Moszer I, Albertini A M, Alloni G, Azevedo V, Bertero M G, Bessieres P, Bolotin A, Borchert S, Borriss R, Boursier L, Brans A, Braun M, Brignell S C, Bron S, Brouillet S, Bruschi C V, Caldwell B, Capuano V, Carter N M, Choi S K, Codani J J, Connerton I F, Danchin A, et al. The complete genome sequence of the gram-positive bacterium Bacillus subtilis. Nature. 390 (1997) 249-56.
43. Lee H W, Pan J G, Lebeault J M. Enhanced L-lysine production in threonine-limited continuous culture of Corynebacterium glutamicum by using gluconate as a secondary carbon source with glucose. Applied Microbiology and Biotechnology 49 (1998) 9-15.
44. Liu S T, Lee L Y, Tai C Y, Hung C H, Chang Y S, Wolfram J H, Rogers R, Goldstein A H. Cloning of an Erwinia herbicola gene necessary for gluconic acid production and enhanced mineral phosphate solubilization in Escherichia coli HB101: nucleotide sequence and probable involvement in biosynthesis of the coenzyme pyrroloquinoline quinone. J Bacteriol 174 (1992) 5814-5819.
45. Matos M V, Coello N. L-lysine production by Corynebacterium glutamicum non growing cells. Acta Cient Venez 50 (1999) 233-239.
46. Matsushita T, Ueda T, Kusaka I. Purification and characterization of Ca2+/H+ antiporter from Bacillus subtilis. Eur J. Biochem. 156 (1986) 95-100.
47. Meesters P A E P, Huijberts G N M, Eggink G. High cell density cultivation of the lipid accumulating yeast Cryptococcus curvatus using glycerol as a carbon source. Applied Microbilogy and Biotechnology 45 (1996) 575-579.
48. Mehta A, Datta A. Oxalate decarboxylase from Collybia velutipes. Purification, characterization, and cDNA cloning. J Biol Chem 266 (1991) 23548-23553.
49. Mostafa H E, Heller K J, Geis A. Cloning of Escherichia coli lacZ and lacY Genes and Their Expression in Gluconobacter oxydans and Acetobacter liquefaciens. Applied and Environmental Microbiology, 68 (2002) 2619-2623.
50. Oeda M. Crystallization of κ-casein. II Crystals of κ-casein-lactate complex. Nihon Jui Chikusan Daigaku Kiyo 27 (1978) 175-177.
51. Ohnishi J, Mitsuhashi S, Hayashi M, Ando S, Yokoi H, Ochiai K, Ikeda M. A novel methodology employing Corynebacterium glutamicum genome information to generate a new L-lysine-producing mutant. Appl Microbiol Biotechnol 58 (2002) 217-223.
52. Pandey A, Nigam P, Soccol C R, Soccol V T, Singh D, Mohan R. Advances in microbial amylases. Biotechnology and Applied Biochemistry 31 (2000) 135-152.
53. Patek M, Bilic M, Krumbach K, Eikmanns B, Sahm H, Eggeling L. Identification and transcriptional analysis of the dapB-ORF2-dapA-ORF4 operon of Corynebacterium glutamicum, encoding two enzymes involved in L-lysine synthesis. Biotechnology Letters 19 (1997) 1113-1117.
54. Pedersen H, Hjort C, Nielsen J. Cloning and characterization of oah, the gene encoding oxaloacetate hydrolase in Aspergillus niger. Mol Gen Genet. 263 (2000) 281-286.
55. Popkin P S, Maas W K. Escherichia coli regulatory mutation affecting lysine transport and lysine decarboxylase. J Bacteriol 141 (1980) 485-492.
56. Prasad G S, Vijayan M. X-ray studies on crystalline complexes involving amino acids and peptides. XXII. Variability in ionization state, conformation and molecular aggregation in the complexes of succinic acid with DL- and L-lysine. Acta Crystallographica B47 (1991) 927-935.
57. Presecan S E, Galinier A, Longin R, Deutscher J, Danchin A, Glaser P, Martin-Verstraete I. Catabolite regulation of the pta gene as part of carbon flow pathways in Bacillus subtilis. Bacteriol 181 (1999) 6889-6897.
58. Pronk J T, Levering P R, Olijve W, Van Dijken J P. Role of NADP-dependent and quinoprotein glucose dehydrogenases in gluconic acid production by Gluconobacter oxydans. Enzyme and Microbial Technology 11 (1989) 160-164.
59. Punt P J, Oliver R P, Dingemanse M A, Pouwels P H, van den Hondel C A. transformation of Aspergillus based on the hygromycin B resistance marker from Escherichia coli. Gene 56 (1987) 117-124.
60. Ratledge C. Regulation of lipid accumulation in oleaginous micro-organisms. Biochemical Society Transactions 30 (2002) 1047-1050.
61. Renno D V, Saunders G, Bull A T, Holt G. The genetic stability of Penicillium chrysogenum transformants in a fermentor. Appl Microbiol Biotechnol 34 (1990) 364-367.
62. Revelles O, Espinosa-Urgel M, Molin S, Ramos J L. The davDT Operon of Pseudomonas putida, Involved in lysine catabolism, is induced in response to the pathway intermediate {delta}-aminovaleric acid. J Bacteriol 186 (2004) 3439-3446.
63. Ruijter G J G, Panneman H, Visser J. Overexpression of phospho-fructokinase and pyruvate kinase in citric acid producing Aspergillus niger. Biochim. Biophys. Acta 1334 (1997) 317-326.
64. Ruijter G J G, van de Vondervoort P J, Visser J. Oxalic acid production by Aspergillus niger: an oxalate-non-producing mutant produces citric acid at pH 5 and in the presence of manganese. Microbiology 145 (1999) 2569-2576.
65. Ruijter G J G, Panneman H, Xu D-B, Visser J. Properties of Aspergillus niger citrate synthase and effects of citA overexpression on citric acid production. FEMS Microbiology Letters 184 (2000) 35-40.
66. Salunke D M, Vijayan M. Crystal structure of the amino acid-vitamin complex lysine pantothenate. Biochimica et Biophysica Acta 798 (1984) 175-179.
67. Saraswathi N T, Siddhartha R, Vijayan M. X-ray studies on crystalline complexes involving amino acids and peptides. XLI. Commonalities in aggregation and conformation revealed by the crystal structures of the pimelic acid complexes of L-arginine and DL-lysine. Acta Crystallographica B59 (2003) 641-646.
68. Shiratsuchi M, Kuronuma H, Kawahara Y, Yoshihara Y, Miwa H, Nakamori S. Simultaneous and high fermentative production of L-lysine and L-glutamic acid using a strain of Brevibacterium lactofermentum. Biosci Biotech biochem 59 (1995) 83-86.
69. Tauch A, Puhler A, Kalinowski J, Thierbach G. Plasmids in Corynebacterium glutamicum and their molecular classification by comparative genomics. Journal of Biotechnology 104 (2003) 27-40.
70. Theilgaard H, van Den Berg M, Mulder C, Bovenberg R, Nielsen J. Quantitative analysis of Penicillium chrysogenum Wis54-1255 transformants overexpressing the penicillin biosynthetic genes. Biotechnol Bioeng 72 (2001) 379-388.
71. Van de Rhee M D, Mendes O, Werten M W, Huizing H J, Mooibroek H. Highly efficient homologous integration via tandem exo-beta-1,3-glucanase genes in the common mushroom, Agaricus bisporus. Curr Genet. 30 (1996) 166-173
72. Van der Rest M E, Lange C, Molenaar D. A heat shock following electroporation induces highly efficient transformation of Corynebacterium glutamicum with xenogeneic plasmid DNA. Applied Microbiology and Biotechnology 52 (1999) 541-545.
73. Van Gulik W M, de Laat W T, Vinke J L, Heijnen J J. Application of metabolic flux analysis for the identification of metabolic bottlenecks in the biosynthesis of penicillin G. Biotechnol Bioeng 20 (2000) 602-618.
74. Venkatraman J, Prabu M M, Vijayan M. X-ray studies on crystalline complexes involving amino acids and peptides. XXXII. Effect of chirality on ionisation state, stoichiometry and aggregation in the complexes of oxalic acid with DL- and L-lysine. Journal of Peptide Research 50 (1997) 77-87.
75. Verma V, Qazi G N, Parshad R, Chopra C L. A fast spheroplast formation procedure in some 2,5-diketo-D-gluconate- and 2-keto-L-gulonate-producing bacteria. Biotechniques 7 (1989) 449-452.
76. Verma V, Qazi G N, Parshad R. Intergeneric protoplast fusion between Gluconobacter oxydans and Corynebacterium species. J Biotechnol 26 (1992) 327-330.
77. Wijayarathna C D, Wachi M, Nagai K. Isolation offtsi and murE genes involved in peptidoglycan synthesis from Corynebacterium glutamicum. Appl Microbiol Biotechnol. 55 (2001) 466-470.
78. Yanase H, Mori N, Masuda M, Kita K, Shimao M, kato N. Pyruvate production by Enterococcus casselifavus a-12 from gluconate in an alkaline medium. Journal of Fermentation and Bioengineering 73 (1992) 287-291.

### APPENDIX 1

Genome Information from the NCBI Website, http://www.ncbi.nlm.nih.gov/entrez, Used for the Design of Degenerate or Specific PCR Primers and Subsequent Gene Isolation.

1a: Sequences Used for Isolating the G. oxydans Lysine Transporter (lysI) Gene.

The gene was isolated using degenerate PCR primers that were designed on the basis of the of applicable (complete) genome sequences at http://www.ncbi.nlm.nih.gov/entrez/: Staphylococcus aureus subsp. aureus N315, gi29165615, NC_{—}002745.2; Mesorhizobium loti MAFF303099, gi/3470324, NC_{—}002678.1; Lactococcus lactis subsp. lactis 111403, gi/5671982, NC_{—}002662.1; Escherichia coli K12, gi/612799, NC_{—}000913.1; Clostridium perfringens str. 13, gi/8308982, NC_{—}003366.1; Corynebacterium diphtheriae NCTC 13129, gi38232642, NC_{—}002935.2; Clostridium acetobutylicum ATCC 824 gi/5893298, NC_{—}003030.1; Escherichia coli O157:H7, gi/5829254, NC_{—}002695.1; Agrobacterium tumefaciens str. C58, gi/5890089, NC_{—}003063.1; Agrobacterium tumefaciens str. C58, gi/5887359, NC_{—}003062.1; Sinorhizobium meliloti 1021, gi/5963753, NC_{—}003047.1; Lactobacillus plantarum WCFS1, gi28376974, NC_{—}004567.1; Sinorhizobium meliloti 1021 plasmid pSymB, gi/6263748, NC_{—}003078.1; Sinorhizobium meliloti 1021 plasmid pSymA, gi/6262453, NC_{—}003037.1; Corynebacterium glutamicum ATCC 13032, gi23308765, NC_{—}003450.2; Bacillus cereus ATCC 14579, gi30018278, NC_{—}004722.1; Streptococcus pneumoniae R6, gi/5902044, NC_{—}003098.1; Bacillus subtilis, gi/6077068, NC_{—}000964.1; Pseudomonas aeruginosa PA01, gi/5595198, NC_{—}002516.1.

1b: Sequences Used for Isolating the Gluconolactonase Gene from G. oxydans.

The gene was isolated using degenerate PCR primers that were designed on the basis of the Zymomonas mobilis (Kanagasundaram and Scopes, 1992) and of other applicable (complete) genome sequences at http://www.ncbi.nlm.nih.gov/entrez/: Mesorhizobium loti MAFF303099 (gi13470324, NC_{—}002678.1), Pirellula sp.1 (gi32470666, NC_{—}005027.1), Bradyrhizobium japonicum USDA 110 (gi27375111, NC_{—}004463.1), Brucella melitensis 16M chromosome I (gi17986284, NC_{—}003317.1), Ralstonia solanacearum GMI1000 plasmid pGMI1000MP (gi17548221, NC_{—}003296.1, Thermoplasma volcanium GSS1 (gi13540831, NC_{—}002689.2), Deinococcus radiodurans R1 chromosome 1 (gi15805042, NC_{—}001263.1), Xylella fastidiosa 9a5c (gi15836605, NC_{—}002488.1), Agrobacterium tumefaciens str. C58 (gi17936711, NC_{—}003305.1), Agrobacterium tumefaciens str. C58 (gi15890089, NC_{—}003063.1), Streptomyces coelicolor A3(2) (gi32141095, NC_{—}003888.3), Sinorhizobium meliloti 1021 plasmid pSymB (gi16263748, NC_{—}003078.1), Sinorhizobium meliloti 1021 plasmid pSymA (gi16262453, NC_{—}003037.1), Rhodopseudomonas palustris CGA009 (gi39933080, NC_{—}005296.1), Bordetella bronchiseptica RB50 (gi33598993, NC_{—}002927.3), Pseudomonas syringae pv. tomato str. DC3000 (gi28867243, NC_{—}004578.1), Xylella fastidiosa Temecula 1 (gi28197945, NC_{—}004556.1), Brucella suis 1330 chromosome II (gi23499767, NC_{—}004311.1), Xanthomonas axonopodis pv. citri str. 306 (gi21240774, NC_{—}003919.1), Xanthomonas campestris pv. campestris str. ATCC 33913 (gi21229478, NC_{—}003902.1), Caulobacter crescentus CB15 (gi16124256, NC_{—}002696.2).

1c: Sequences Used for Isolating the Gluconokinase Gene from G. oxydans

Sequences used for isolating the gluconokinase gene from G. oxydans were obtained using DNA sequence information from Fujita and Fujita, 1987; Yanase et al., 1992; Izu et al., 1994; Isturiz T, Celaya J., 1997 and other applicable (complete) genome sequences at http://www.ncbi.nlm.nih.gov/entrez/: Staphylococcus aureus subsp. aureus N315 (gi29165615, NC_{—}002745.2); Bacillus anthracis str. Ames 0581 (gi47525254, NC_{—}007530.1); Mesorhizobium loti MAFF303099 (gi13470324, NC_{—}002678.1); Escherichia coli K12 (gi16127994, NC_{—}000913.1); Photorhabdus luminescens subsp. laumondii TTO1 (gi37524032, NC_{—}005126.1); Mycobacterium bovis subsp. bovis AF2122/97 (gi31791177, NC_{—}002945.3); Nostoc sp. PCC 7120 (gi17227497, NC_{—}003272.1); Yersinia pestis biovar Medievalis str. 91001 (gi45439865, NC_{—}005810.1); Bradyrhizobium japonicum USDA 110 (gi27375111, NC_{—}004463.1); Ralstonia solanacearum GMI1000 (gi17544719, NC_{—}003295.1); Deinococcus radiodurans R1 chromosome 1 (gi15805042, NC_{—}001263.1); Salmonella enterica subsp. enterica serovar Typhi str. CT18 (gi16758993, NC_{—}003198.1); Staphylococcus aureus subsp. aureus Mu50 (gi15922990, NC_{—}002758.1); Neisseria meningitidis MC58 (gi15675948, NC_{—}003112.1); Corynebacterium diphtheriae NCTC 13129 (gi38232642, NC_{—}002935.2); Bifidobacterium longum NCC2705 (gi23464628, NC_{—}004307.1); Escherichia coli O157:H7 (gi15829254, NC_{—}002695.1); Gloeobacter violaceus PCC 7421 (gi37519569, NC_{—}005125.1); Agrobacterium tumefaciens str. C58 (gi17933925, NC_{—}003304.1); Agrobacterium tumefaciens str. C58 (gi15887359, NC_{—}003062.1); Streptomyces avermitilis MA-4680 (gi29826540, NC_{—}003155.2); Lactobacillus plantarum WCFS1(gi28376974, NC_{—}004567.1); Streptomyces coelicolor A3(2) (gi32141095, NC_{—}003888.3); Sinorhizobium meliloti 1021 plasmid pSymB (gi16263748, NC_{—}003078.1); Rhodopseudomonas palustris CGA009 (gi39933080, NC_{—}005296.1); Salmonella typhimurium LT2 (gi16763390, NC_{—}003197.1); Vibrio vulnificus YJO16 chromosome I (gi37678184, NC_{—}005139.1); Lactobacillus johnsonii NCC 533 (gi42518084, NC_{—}005362.1); Oceanobacillus iheyensis HTE831(gi23097455, NC_{—}004193.1); Chromobacterium violaceum ATCC 12472 (gi34495455, NC_{—}005085.1); Shigella flexneri 2a str. 2457T (gi30061571, NC_{—}004741.1); Bacillus cereus ATCC 14579 (gi30018278, NC_{—}004722.1); Salmonella enterica subsp. enterica serovar Typhi Ty2 (gi29140543, NC_{—}004631.1); Pseudomonas syringae pv. tomato str. DC3000 (gi28867243, NC_{—}004578.1); Staphylococcus epidermidis ATCC 12228 (gi27466918, NC_{—}004461.1); Vibrio vulnificus CMCP6 chromosome I (gi27363490, NC_{—}004459.1); Escherichia coli CFT073 (gi26245917, NC_{—}004431.1); Pseudomonas putida KT2440 (gi26986745, NC_{—}002947.3); Shigella flexneri 2a str. 301 (gi24111450, NC_{—}004337.1); Yersinia pestis strain C092 (gi16120353, NC_{—}003143.1); Neisseria meningitidis serogroup A strain Z2491 (gi15793034, NC_{—}003116.1); Yersinia pestis KIM (gi22123922, NC_{—}004088.1); Xanthomonas axonopodis pv. citri str. 306 (gi21240774, NC_{—}003919.1); Xanthomonas campestris pv. campestris str. ATCC 33913 (gi21229478, NC_{—}003902.1); Escherichia coli O157:H7 EDL933 (gi16445223, NC_{—}002655.2); Pseudomonas aeruginosa PA01 (gi15595198, NC_{—}002516.1); Vibrio cholerae O1 biovar eltor str. N16961 chromosome I (gi15640032, NC_{—}002505.1); Vibrio parahaemolyticus RIMD 2210633 chromosome I (gi28896774, NC_{—}004603.1); Corynebacterium efficiens YS-314 (gi25026556, NC_{—}004369.1); Staphylococcus aureus subsp. aureus MW2 (gi21281729, NC_{—}003923.1); Schizosaccharomyces pombe chromosome I (gi19113674, NC_{—}003424.1); Caenorhabditis elegans chromosome V (gi32967584, NC_{—}003283.3); Caenorhabditis elegans chromosome II (gi25168254, NC_{—}003280.2).

1d: Sequences Used for Isolating the P. chrysogenum and A. niger Genes Involved in Oxalate Biosynthesis, Breakdown and Export.

Applicable (complete) genome sequences at http://www.ncbi.nlm.nih.gov/entrez/: Streptomyces coelicolor A3(2), gi32141095, NC_{—}003888.3; Arabidopsis thaliana chromosome 1, gi42592260, NC_{—}003070.5; Neisseria meningitidis serogroup A strain Z2491, gi15793034, NC_{—}003116.1; mgap007xE14f.b Magnaporthe grisea Ap Uni-Zap XR Library Magnaporthe grisea cDNA clones; Arabidopsis thaliana chromosome 1, gi12063420, gbAE005172.1; Flammulina velutipes oxalate decarboxylase (OXDC) gene, gi6468005, gbAF200683.1; AF200683 Hordeum vulgare mRNA for oxalate oxidase (HvOxOa) gi2266667, emb, Y14203.1; HVY14203[2266667] Hvulgare mRNA for oxalate oxidase-like protein; gi1070357, emb, X93171.1.; oxalate decarboxylase Agrobacterium tumefaciens str. C58 Chromosome: linearGeneID: 1136645; Oxalate decarboxylase Bacillus cereus ATCC 14579, GeneID: 1204732; oxalate decarboxylase Bradyrhizobium japonicum USDA 110, GeneID: 1049607; Oxalate decarboxylase Bacillus cereus ATCC 14579, GeneID: 1203380; solute carrier family 26 (sulfate transporter), member 1, Mus musculus; sulfate anion transporter-1Chromosome: 5; Location: 5 FGeneID: 231583; alpha-1-microglobulin/bikunin precursor Homo sapiens.

### APPENDIX 2

Published and Commercially Available Complexes Between Amino Acids and Organic Acids.

TABLE 3
Examples of basic amino acid - carboxylic
acid complexes retreived from literature.

| Carboxylic | Amino acid | | |
|---|---|---|---|
| acid | Arginine | Lysine | Histidine |

Monocarboxylic acid

| | | | |
|---|---|---|---|
| Formic acid | Suresh et al., 1994a | | Suresh & Vijayan, |
| | | | 1995 |
| Acetic acid | Suresh & Vijayan, | Suresh & Vijayan, | Suresh et al., 1994b |
| | 1983b; Soman et al., | 1983a; Soman et al., | |
| | 1989 | 1989 | |

Dicarboxylic acid

| | | | |
|---|---|---|---|
| Oxalic acid | Chandra et al., 1998 | Venkatraman et al., | Prabu et al., 1996 |
| | | 1997 | |
| Malonic acid | Saraswathi & Vijayan, | | Saraswathi & Vijayan, |
| | 2002b | | 2002a |
| Succinic acid | Prasad & Vijayan, | Prasad & Vijayan, | Prasad & Vijayan, |
| | 1990 | 1991 | 1993 |
| Glutaric acid | Saraswathi & Vijayan, | Saraswathi et al., 2001 | Saraswathi & Vijayan, |
| | 2001 | | 2001 |
| Pimelic acid | Saraswathi et al., 2003 | Saraswathi et al., 2003 | |

Hydroxy-monocarboxylic acid

| | |
|---|---|
| Glycolic acid | Suresh & Vijayan, |
| | 1996 |

Unsaturated dicarboxylic acid

| | | |
|---|---|---|
| Maleic acid | Pratap et al., 2000 | Pratap et al., 2000 |

Acidic amino acid

| | | | |
|---|---|---|---|
| Aspartic acid | Soman et al., 1990 | Bhat & Vijayan, 1976 | Bhat & Vijayan, 1978 |
| | | Soman et al., 1988 | |
| Glutamic acid | Bhat & Vijayan, 1977 | Soman et al., 1988 | |
| | Soman et al., 1990 | | |

Vitamin

| | |
|---|---|
| Pantothenatic | Salunke & Vijayan, |
| acid (Vitamin | 1984 |
| B5) | |

On the web-site http://www.buyersguidechem.info/AnameEin.php (directory of chemicals and chemical suppliers), examples of commercially available organic salts are: arginine.acetate, lysine.acetate, ornithine.acetate, lysine.succinate, arginine.alpha-ketoglutarate, ornithine.alpha-ketoglutarate, arginine.aspartate, arginine.glutamate, lysine.glutamate, ornithine.aspartate.

1. A method for the production of at least a first fermentation product and at least a second fermentation product by conversion of a substrate by a production organism into the first fermentation product and the second fermentation product, wherein the organism comprises a first metabolic pathway for conversion of the substrate into the first fermentation product and a second metabolic pathway for conversion of the substrate into the second fermentation product, wherein the first fermentation product is in a more reduced state than the substrate and the second fermentation product is in a more oxidised state than the substrate, wherein the first and second pathways are complementary in that their concurrent action in the organism allows recycling of reducing power in the organism, wherein the second fermentation product is in a less oxidised state than the final oxidation products CO₂ and H₂O and wherein the combined yields of the first and second fermentation products on substrate on a C-molar basis is at least 40%.

2. A method according to claim 1, wherein during the conversion of the substrate into the first and second fermentation product, the organism consumes less than 30% of the amount of oxygen on an O₂/C-molar basis related to the substrate consumed.

3. A method according to claim 1, wherein the biomass yield on substrate is less than 30% on a C-molar basis related to the substrate consumed.

4. A method according to claim 1, wherein the first fermentation product comprises more than one compound, each of which being in a more reduced state than the substrate.

5. A method according to claim 1, wherein second fermentation product comprises more than one compound, each of which being in a more oxidised state than the substrate.

6. A method according to claim 1, wherein the first fermentation product is a weak alkaline compound or a salt thereof or derivative thereof and the second fermentation product is a weak acidic compound or a salt thereof or derivative thereof.

7. A method according to claim 1, wherein a first and a second fermentation product are capable of forming an insoluble complex or insoluble salt under the fermentation conditions applied.

8. A method according to claim 1, wherein at least part of the first or the second metabolic pathway is not naturally present or active in the organism.

9. A method according to claim 8, wherein the first or second metabolic pathway is introduced or activated in the organism by a genetic modification of the organism.

10. A method according to claim 1, wherein the organism is a microorganism or an in vitro cultured cell of a higher organism.

11. A method for the production of at least a first fermentation product and at least a second fermentation product by conversion of a substrate by a first production organism into the first fermentation product and by conversion of a substrate by a second production organism into the second fermentation product, wherein the first and second production organism are co-fermented and wherein the first fermentation product is a weak alkaline compound or a salt thereof or derivative thereof and the second fermentation product is a weak organic acid or a salt thereof or derivative thereof.

12. A method for the production of at least a first fermentation product and at least a second fermentation product by conversion of a substrate by a first production organism into the first fermentation product and by conversion of a substrate by a second production organism into the first fermentation product, wherein the first and second production organisms are co-fermented and wherein a first and a second fermentation product are capable of forming an insoluble complex or insoluble salt under the fermentation conditions applied.

13. A method according to claim 12, wherein the first fermentation product is a weak alkaline compound or a salt thereof or derivative thereof and the second fermentation product is a weak acidic compound or a salt thereof or derivative thereof.

14. A method according to claim 11, wherein the first production organism is cultured in a first fermenter and the second production organism is cultured in a second fermenter, wherein the first and second fermenters are run in parallel and are connected through one or more connecting means that comprise a micro-sieve, wherein soluble medium components are circulated between the first and second fermenters through the connecting means comprising the micro-sieve while the micro-sieve prevents circulation of the production organisms.

15. A method according to claim 14, wherein the connecting means further connect to a vessel that is separated from the first and second fermenters by the micro-sieve.

16. A method according to claim 15, wherein in the vessel a condition is applied that promote the formation of an insoluble complex or an insoluble salt between the first and second fermentation products.

17. A method according to claim 16, wherein the condition that promotes the formation of an insoluble complex or an insoluble salt between the first and second fermentation products is one or more conditions selected from:
a) a temperature that is lower than the temperature in the first and second fermenters;
b) a pH that differs from the pH in the first and second fermenters; and
c) a concentration of helper molecule that promotes the formation of an insoluble complex or insoluble salt between the first and second fermentation products.

18. A method according to claim 17, wherein the helper molecule is a metal ion or a water-miscible organic solvent.

19. A fermentation system comprising at least two different fermentation volumes, wherein each fermentation volume comprises at least one fermenter, wherein the different fermentation volumes are connected through connecting means comprising a micro-sieve that allows circulation of soluble medium components between different fermenter volumes while preventing circulation of cells of the production organism between the fermentation volumes.

20. A fermentation system according to claim 19, wherein the connecting means further connect to a vessel that is separated from the different fermenter volumes by the micro-sieve.
